SEQUENCE LISTING (26 sequences)

SEQ ID NO: 1 - DNA, 19 bp, Homo sapiens
agcccagaacactggtctc

SEQ ID NO: 2 - DNA, 21 bp, Homo sapiens
actcaggatttcaatggtgcc

SEQ ID NO: 3 - DNA, 23 bp, Homo sapiens
tatgagtgggaccaaaagtaccg

SEQ ID NO: 4 - DNA, 22 bp, Homo sapiens
gggattgaaggttaggctctgg

SEQ ID NO: 5 - DNA, 22 bp, Homo sapiens
tcctgaacctgagtagagacac

SEQ ID NO: 6 - DNA, 19 bp, Homo sapiens
tgctgcttgtagtggctgg

SEQ ID NO: 7 - DNA, 20 bp, Homo sapiens
ctccagggacaggatatgga

SEQ ID NO: 8 - DNA, 20 bp, Homo sapiens
tctttcaacacgcaggacag

SEQ ID NO: 9 - DNA, 21 bp, Mus musculus
aggtcggtgtgaacggatttg

SEQ ID NO: 10 - DNA, 23 bp, Mus musculus
tgtagaccatgtagttgaggtca

SEQ ID NO: 11 - DNA, 22 bp, Mus musculus
agcttgtctcctgaaaatcgag

SEQ ID NO: 12 - DNA, 22 bp, Mus musculus
aggtttgattcaggcagatgtt

SEQ ID NO: 13 - DNA, 20 bp, Mus musculus
agggtctacggggcaatttc

SEQ ID NO: 14 - DNA, 21 bp, Mus musculus
tcacagtccgtttccggagtt

SEQ ID NO: 15 - DNA, 20 bp, Mus musculus
ggtctcaacccccagctagt

SEQ ID NO: 16 - DNA, 23 bp, Mus musculus
gccgatgatctctctcaagtgat

SEQ ID NO: 17 - DNA, 21 bp, Mus musculus
gctcttactgactggcatgag

SEQ ID NO: 18 - DNA, 20 bp, Mus musculus
cgcagctctaggagcatgtg

SEQ ID NO: 19 - DNA, 19 bp, Mus musculus
cctggctcttgcttgcctt

SEQ ID NO: 20 - DNA, 21 bp, Mus musculus
ggtcttgtgtgatgttgctca

SEQ ID NO: 21 - DNA, 23 bp, Mus musculus
atgagtttttcccttatggggac

SEQ ID NO: 22 - DNA, 21 bp, Mus musculus
gctggaagttggacacctcaa

SEQ ID NO: 23 - DNA, 22 bp, Mus musculus
tcagcagacgaatcaatacagc

SEQ ID NO: 24 - DNA, 22 bp, Mus musculus
tcgctcaacacttggactttct

SEQ ID NO: 25 - DNA, 22 bp, Mus musculus
gctgcagaagggagaaatcacg

SEQ ID NO: 26 - DNA, 30 bp, Mus musculus
ggagttggaatacttcattctaggtctcat

(19) United States
(12) Patent Application Publication
(10) Pub. No.: US 2023/0100173 A1
(43) Pub. Date: Mar. 30, 2023

(54) A TOPICAL COMPOSITION COMPRISING AN EXTRACT OF COMBINED HERBS COMPRISING LONGANAE ARILLUS FOR TLSP INHIBITION AND THE TREATMENT OR ALLEVIATION OF SKIN INFLAMMATORY DISEASE AND THE USE THEREOF

(71) Applicants: Ok Nam PARK, Seoul (KR); MEDIHELPLINE CO., LTD, Seoul (KR)

(72) Inventor: Ok Nam PARK, Seoul (KR)

(21) Appl. No.: 17/911,399

(22) PCT Filed: Mar. 10, 2021
(86) PCT No.: PCT/KR2021/002964

§ 371 Date: Sep. 13, 2022

(30) Foreign Application Priority Data
Mar. 13, 2020 (KR) 10-2020-0031482
Feb. 19, 2021 (KR) 10-2021-0022675

(51) Int. Cl. A61K 36/77; A61K 9/00; A61K 36/69; A61K 36/236; A61K 8/9789; A61P 29/00; A61P 17/00; A61Q 19/00

(52) U.S. Cl. CPC A61K 36/77; A61K 9/0014; A61K 36/69; A61K 36/236; A61K 8/9789; A61P 29/00; A61P 17/00; A61Q 19/00; A61K 2236/333; A61K 2236/35

(57) ABSTRACT

The present invention is related to a topical pharmaceutical composition and cosmetic composition comprising a combined herb extract of Longanae Arillus, Ligustici Tenuissimi Rhizoma and Polygalae radix as an active ingredient to inhibit TLSP (thymic stromal lymphopoietin) cytokine expression or to treat and alleviate skin inflammatory diseases.

### TECHNICAL FIELD

The present invention relates to a topical composition comprising the extract of combined herbs comprising Longanae Arillus for TSLP inhibition and for the treatment or alleviation of skin inflammatory disease and the use thereof.

### BACKGROUND ART

Chronic recurrent dermatological inflammatory diseases such as atopic dermatitis (atopic dermatitis) and psoriasis are very difficult to treat because of the wide variety of causes.

In particular, in some patients with atopic dermatitis, the filaggrin gene mutates, causing skin barrier damage, causing serious inflammatory reactions due to bacterial infections or infection of foreign substances derived from ticks.

In addition to these genetic factors, the decreased water retention due to reduced ceramide in the stratum corneum and the abnormal elimination of keratinocytes due to increased pH on the skin surface can be a factor in determining the severity of skin diseases along with impaired skin barrier function. In addition, most of these skin diseases are concentrated in early childhood and adolescence, resulting in emotional and educational social problems.

In particular, the number of scratches caused by unbearable itching accompanying these skin diseases increases, which further exacerbates the symptoms. Recently, various cytokines such as interleukin and thymic stromal lymphopoietin (TSLP) in keratinocytes of the skin have been known to increase its expression and stimulate the sensory nerves present under the skin, causing itching as well as dermatitis (Mack et al., 2018, Trends Immunol. 2018 December; 39(12): 980-991)

An inflammatory response is a mechanism to repair and regenerate the damaged area when some invasion resulting in any substrate change, such as physical action, a contact of various chemical substance or bacterial infections, in living organisms or tissues, has been occurred.

Upon the foreign stimuli, various vascular active substances such as inflammatory components are locally released, which increases vascular permeability, causing inflammation, however, inconsistent inflammatory reactions promote mucosal damage, resulting causes various the other diseases. in some case (Willoughby D A. (1975) Human arthritis applied to animal models. Towards a better therapy. Annals of the Rheumatic Disease. 34, 471-4781.

Proinflammatory cytokines as inflammatory indicators, include tumor necrosis factor-alpha (TNF-α), interleukin-1beta (IL-1(3), interleukin-6 (IL-6), monocyte chemoattractant protein-1 (MCP1) etc (Yun H J, Heo S K, Yi H S, Kim C H, Kim B W and Park S D. (2008) Anti-inflammatory effect of injinho-tang in Raw264.7 Cells. Kor. J. Herbology. 23(2), 169-178).

At present, various antihistamine or steroids such as an injection and ointment form of cortisol, prednisolone, methylprednisolone, dexamethasone etc, are commonly used for the treatment of atopic dermatitis, however, it dosed not provides with satisfactory efficacy. In particular, the therapy with steroids induce more severe hypersensitivity due to the enlarged capillaries and thinned skin layers, and furthermore, if steroid application is suspended, resulting steroid rebound causes to show more severe symptom.

Accordingly, there has been still needed to develop more effective drug and cosmetics in treating and alleviating inflammatory skin diseases such as atopic dermatitis from natural resources with low side effects than conventionally used drugs till now.

Longanae Arillus, a seed coat of Dimocarpus longan, Euphoria longan or the same species belonged to Sapindaceae has been reported to contain a glucose, fructose, protein etc and to show cardio-protective effect, appetite stimulating effect etc (Chung B. S et al, Dohaehyangyakdaesajeon, youngrimsa, 2^{nd} Ed. p 197-198, 1998).

Ligustici Tenuissimi Rhizoma, a rhizoma or root of Ligusticum tenuissimum Kitagawa, Ligusticum sinense Oliv, Ligusticum jeholense Nakai et Kitagawa or the same species belonged to Umbelliferae has been reported to contain a cnidilide, 3-butyl phthalide etc and to show anti-bacterial effect etc (Chung B. S et al, Dohaehyangyakdaesajeon, youngrimsa, 2^{nd} Ed. P428-429, 1998).

Polygalae radix, a root of Polygala tenuifolia Willd., or the same species belonged to Polygalaceae has been reported to contain various sanponis and to show expectorant activity, anti-bacterial effect etc (Chung B. S et al, Dohaehyangyakdaesajeon, youngrimsa, 2nd Ed. P798-799, 1998).

However, there has been not reported or disclosed on the preventing or alleviating activity of a topically applied extract of combined herbs of Longanae Arillus, Ligustici Tenuissimi Rhizoma and Polygalae radix showing potent treating effect on skin inflammatory diseases in any of above cited literatures, and the disclosures of which are incorporated herein by reference.

### DISCLOSURE OF INVENTION

### Technical Problem

To investigate the anti-inflammatory effect of a combined herb extract of Longanae Arillus, Ligustici Tenuissimi Rhizoma and Polygalae radix, the inventors of present invention have intensively carried out various experiments including in vitro experiments such as the inhibitory test on the expression of cytokines involved in skin inflammation (RPLPO, TSLP, GM-CSF and IL-1beta) (Experimental Example 1); as well as in vivo experiments such as the inhibitory effect on the atopic dermatitis using by BALB/C mice (Experimental Example 2); the inhibition test of expression of various cytokines involved in skin inflammation (GADPH, TSLP, GM-CSF, IL-4, IL10, IL-13, IL-31 and IL-33) using test animal (Experimental Example 3); the inhibition test of TSLP cytokine expression using test animal (Experimental Example 4). As a result of these investigations, the inventors finally completed the present invention by confirming that inventive combined herb extract strongly inhibited and alleviated TLSP (thymic stromal lymphopoietin) cytokine expression or to treat and alleviate skin inflammatory diseases.

### Solution to Problem

The technical solution to solve the problem of the background art is for the development of novel herb formulation for treating and preventing a skin inflammation disease.

Accordingly, it is an object of the present invention to provide a topical pharmaceutical composition comprising a combined herb extract of Longanae Arillus, Ligustici Tenuissimi Rhizoma and Polygalae radix as an active ingredient to inhibit TLSP (thymic stromal lymphopoietin) cytokine expression or to treat and alleviate skin inflammatory diseases.

The term “combined herb extract” defined herein comprises the combined herb extract, i.e., (a) combined herb extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50:0.1-50 weight part (w/w), more preferably, 0.1-10:0.1-10:0.1-10 weight part (w/w), more and more preferably, 1-5:1-5:1-5 weight part (w/w), most preferably, 1-3:1-3: 1-3 weight part (w/w);

or (b) the combination of each extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50: 0.1-50 weight part (w/w), more preferably, 0.1-10:0.1-10:0.1-10 weight part (w/w), more and more preferably, 1-5:1-5:1-5 weight part (w/w), most preferably, 1-3:1-3: 1-3 weight part (w/w) in the present invention.

it is an another object of the present invention to provide a TLSP expression inhibitor comprising a combined herb extract of Longanae Arillus, Ligustici Tenuissimi Rhizoma and Polygalae radix as an active ingredient in an amount to inhibit TLSP (thymic stromal lymphopoietin) cytokines.

The term “extract” disclosed herein comprises the extract which can be extracted with at least one solvent selected from water, C₁-C₄ lower alkyl alcohol such as methanol, ethanol, propanol, butanol, etc, acetone, ethyl acetate, chloroform, hexane, butyleneglycol, propyleneglycol or glycerin, preferably, water, methanol, ethanol, more preferably, water or 10-90% (v/v) ethanol in water, most preferably, water or 20-80% (v/v) ethanol in water.

The term “skin inflammatory diseases” disclosed herein comprises the disease selected from group of pruritus caused by aging or atopy, chronic relapsing dermatitis such as atopic dermatitis, psoriasis, and the like; contact dermatitis, seborrheic dermatitis, neurodermatitis, xeroderma, erythema, inflammatory dermatitis, psoriasis, or atopic disease; preferably, pruritus caused by aging or atopy resulting from increased expression of the cell cytokines, chronic relapsing dermatitis such as atopic dermatitis, psoriasis, and the like; contact dermatitis, seborrheic dermatitis, neurodermatitis, xeroderma, erythema, inflammatory dermatitis, psoriasis, or atopic disease; more preferably, pruritus caused by aging or atopy resulting from increased expression of the cell TLSP cytokines, chronic relapsing dermatitis such as atopic dermatitis, psoriasis, and the like; contact dermatitis, seborrheic dermatitis, neurodermatitis, xeroderma, erythema, inflammatory dermatitis, psoriasis, or atopic disease.

The term, “anti-Inflammation” disclosed herein, not limited thereto, means all the mechanism to inhibit various inflammation.

Inflammation is part of the complex biological response of body tissues to harmful stimuli, such as pathogens, damaged cells, or irritants, and the nonspecific immune response such as heat, pain, redness, swelling, etc is called as “inflammatory response”

Inflammation can be classified as (a) acute inflammation, the initial response of the body to harmful stimuli, is achieved by the increased movement of plasma and leukocytes (especially granulocytes) from the blood into the injured tissues and then a series of biochemical events propagates and matures the inflammatory response, involving the local vascular system, the immune system, and various cells within the injured tissue and (b) Prolonged inflammation, known as chronic inflammation, leading to a progressive shift in the type of cells present at the site of inflammation, such as mononuclear cells, and being characterized by simultaneous destruction and healing of the tissue from the inflammatory process.

Generally, the macrophage in damaged cell excrete various cytokines, which activates T lymphocyte and mast cell, a lymphocyte, releases various histamines, which initiate internal barrier response, resulting in inducing inflammation of the inflected cells. Accordingly, the expressed level of cell cytokines may be used as an indicator of the activation of inflammatory response (the other aspects, anti-inflammatory activity). The “anti-inflammatory activity” disclosed herein denotes the inhibitory activity against various skin inflammation.

Cytokines means all the immunological substances including chemokines, interferons, interleukins, lymphokines, and tumour necrosis factors produced by a broad range of cells, including immune cells like macrophages, B lymphocytes, T lymphocytes and mast cells, as well as endothelial cells, fibroblasts, and various stromal cells, which are released through immunological progress caused by the infiltration of various pathogen such as virus etc.

Generally, cytokines are released at initial stage of infection, however, released constantly where the immune system becomes extraordinarily activated. When the high-level of cytokines are released for a long time such as more than week, we called as “a cytokine storm”, which is a physiological reaction in which the innate immune system causes an uncontrolled and excessive release of pro-inflammatory signaling molecules called cytokines and it exacerbates the inflammation resulting from the extremely abundant homing of immune cells to the inflected area, causes to blood extravasation through the loosening of blood vessel and severely to death. The term “the inhibitory activity of cytokine expression” disclosed herein can be interpreted as a prevention, treatment or improvement of cytokine storm.

The term “cytokine” disclosed herein, not intended to limit thereto, comprises various cytokine involved in dermatitis, such as atopic dermatitis, specifically, the cytokine selected from group of TLSP (thymic stromal lymphopoietin), colony stimulating factor (CSF) such as GM-CSF (granulocyte-macrophage colony stimulating factor), M-CSF (macrophage colony stimulating factor), G-CSF (granulocyte colony stimulating factor) and the like, interleukins such as interleukin-1 (IL-1), IL-4, IL-10, IL-12, IL-13, IL-31, IL-33 and the like, tumor necrosis factor alpha (TNF-α), interferon gamma (IFNγ) etc,

An inventive extract may be prepared in accordance with the following preferred embodiment.

For the present invention, above described extract can be prepared by follows;

The term “combined herb extract of Longanae Arillus, Ligustici Tenuissimi Rhizoma and Polygalae radix” defined herein can be prepared by the procedure comprising the steps; of slicing and washing Longanae Arillus, Ligustici Tenuissimi Rhizoma and Polygalae radix” to use as a basic extraction material at 1st step; mixing together thoroughly with the mixed ratio based on the dried weight of Longanae Arillus, Ligustici Tenuissimi Rhizoma and Polygalae radix (w/w) ranging from 0.01-100:0.01-100:0.01-100 weight part (w/w), preferably, 0.1-50:0.1-50:0.1-50 weight part (w/w), more preferably, 0.1-10:0.1-10:0.1-10 weight part (w/w), more and more preferably, 1-5:1-5:1-5 weight part (w/w), most preferably, 1-3:1-3:1-3 weight part (w/w) to afford the mixed material at 2^{nd} step; adding 1-20 fold volume (v/w), preferably, 4-8 fold volume (v/w) of extracting solvent selected from the group consisting of water, C₁-C₄ lower alkyl alcohol such as methanol, ethanol, propanol, butanol, etc, acetone, ethyl acetate, chloroform, hexane, butyleneglycol, propyleneglycol or glycerin, preferably, water, methanol, ethanol, more preferably, water or 10-90% (v/v) ethanol in water, most preferably, water or 20-80% (v/v) ethanol in water to the mixed material at 3rd step; extracting each solution with the extraction method by the extraction with hot water extraction, cold water extraction, reflux extraction or ultra-sonication extraction, preferably, hot water extraction at the temperature ranging from 50° C. to 120° C., preferably, about 80° C. to 100° C., for the period ranging from 1 to 24 hours, preferably, 2 to 12 hours at 4th step; repeating the above-described extraction process to collect each filtrate with filtration, drying through freeze drying, natural air drying or hot air drying process, preferably freeze drying process to obtain the combined herb extract of Longanae Arillus, Ligustici Tenuissimi Rhizoma and Polygalae radix of the present invention.

It is another object of the present invention to provide a process for preparing the combined herb extract of Longanae Arillus, Ligustici Tenuissimi Rhizoma and Polygalae radix of the present invention.as described above.

It is another object of the present invention to provide a topical pharmaceutical composition comprising a combined herb extract of Longanae Arillus, Ligustici Tenuissimi Rhizoma and Polygalae radix prepared by the above-described process, as an active ingredient to inhibit TLSP (thymic stromal lymphopoietin) cytokine or to treat and alleviate skin inflammatory diseases.

In accordance with another aspect of the present invention, there is also provided a method of inhibiting TLSP (thymic stromal lymphopoietin) cytokine, treating or alleviating skin inflammatory diseases in a mammal comprising topically administering to said mammal an effective amount of the combined herb extract of Longanae Arillus, Ligustici Tenuissimi Rhizoma and Polygalae radix and pharmaceutically acceptable carrier thereof.

In accordance with the other aspect of the present invention, there is also provided a use of the combined herb extract of Longanae Arillus, Ligustici Tenuissimi Rhizoma and Polygalae radix for manufacture of topical preparation employed for inhibiting TLSP (thymic stromal lymphopoietin) cytokine, treating or alleviating skin inflammatory diseases in mammals including human as an active ingredient.

The inventive composition may additionally comprise conventional carrier, adjuvants or diluents in accordance with a using method. It is preferable that said carrier is used as appropriate substance according to the usage and application method, but it is not limited. Appropriate diluents are listed in the written text of Remington's Pharmaceutical Science (Mack Publishing co, Easton Pa.).

Hereinafter, the following formulation methods and excipients are merely exemplary and in no way limit the invention.

The inventive composition according to the present invention can be provided as a topical pharmaceutical composition containing pharmaceutically acceptable carriers, adjuvants or diluents, e.g., lactose, dextrose, sucrose, sorbitol, mannitol, xylitol, erythritol, maltitol, starches, acacia rubber, alginate, gelatin, calcium phosphate, calcium silicate, cellulose, methyl cellulose, polyvinyl pyrrolidone, water, methylhydroxy benzoate, propylhydroxy benzoate, talc, magnesium stearate and mineral oil. The formulations may additionally include fillers, anti-agglutinating agents, lubricating agents, wetting agents, flavoring agents, emulsifiers, preservatives and the like. The compositions of the invention may be formulated so as to provide quick, sustained or delayed release of the active ingredient after their administration to a patient by employing any of the procedures well known in the art.

For topical administration, the inventive extract of the present invention can be formulated in the form of ointments and creams including topical preparation such as cream, gel, patch, spray solution, emulsion, ointment, lotion, liniment, balm, solution, suspension, pack, paste, aerosol, cataplasma and the like.

The inventive composition of the present invention in pharmaceutical dosage forms may be used in the form of their pharmaceutically acceptable salts, and also may be used alone or in appropriate association, as well as in combination with other pharmaceutically active compounds.

The desirable dose of the inventive extract or composition varies depending on the condition and the weight of the subject, severity, drug form, route and period of administration, and may be chosen by those skilled in the art. However, in order to obtain desirable effects, it is generally recommended to topically administer at the amount ranging 0.01-10 g/kg, preferably, 1 to 5 g/kg by weight/day of the inventive extract of the present invention. The dose may be administered in a single or multiple doses per day. In terms of composition, the inventive extract should be present between 0.01 to 80% by weight, preferably 0.5 to 50% by weight based on the total weight of the composition.

The present inventors demonstrated that the anti-inflammatory effects of inventive composition is potent by accomplishing in vitro experiments such as the inhibitory test on the expression of cytokines involved in skin inflammation (RPLPO, TSLP, GM-CSF and IL-1beta) (Experimental Example 1); as well as in vivo experiments such as the inhibitory effect on the atopic dermatitis using by BALB/C mice (Experimental Example 2); the inhibition test of expression of various cytokines involved in skin inflammation (GADPH, TSLP, GM-CSF, IL-4, IL-10, IL-13, IL-31 and IL-33) using test animal (Experimental Example 3); the inhibition test of TSLP cytokine expression using test animal (Experimental Example 4), therefore, it is confirmed that inventive combined extract is very useful in the alleviation or treatment of skin inflammation as a form of topical medicament or cosmetic composition.

It is the other object of the present invention to provide a cosmetic composition comprising the combined herb extract of Longanae Arillus, Ligustici Tenuissimi Rhizoma and Polygalae radix as an active ingredient in an amount effective to inhibit TLSP (thymic stromal lymphopoietin) cytokine or to treat and alleviate skin inflammatory diseases.

It is preferable that the present cosmetic composition contains 0.001-40%, more preferably, 0.01-10% by the weight of the inventive composition based on the total weight of the composition. The other components may be a mixture of the ingredients of a conventional cosmetic composition well known in the art.

Cosmetic formulations containing above composition may be prepared in any form such as skin lotion, skin softener, skin toner, astringent, lotion, milk lotion, moisture lotion, nutrient lotion, massage cream, nutrient cream, moisture cream, hand cream, foundation, essence, nutrient essence, pack, cleansing foam, cleansing lotion, cleansing cream, body lotion, body cleanser, treatment, beauty solution and the like.

Hereinafter, the following formulation methods and excipients are merely exemplary and in no way limit the invention.

The cosmetic composition of the present invention can comprises additional additives selected from the group consisting of water soluble vitamin, lipid soluble vitamin, peptide polymer, polysaccharide polymer, sphingolipid and sea-weed extract.

Preferable water soluble vitamins are any one which can be mixed with cosmetic, however, various vitamin such as vitamin B₁, B₂, B₆, pyridoxine, pyridoxine HCl, vitamin B₁₂, pantothenic acid, nicotinic acid, nicotinamide, folic acid, vitamin C, vitamin H etc, the salt thereof such as thiamin HCl salt, ascorbic acid Na salt etc or their derivatives such as ascorbic acid-2-phosphonic acid Na salt, ascorbic acid-2-phosphonic acid Mg salt are preferable and those can be obtained by conventional method such as microbial conversion method, purification method from the microbial cultivates, enzymatic method or chemical synthetic method.

Preferable lipid soluble vitamins are any one which can be mixed with cosmetic, however, various vitamin such as vitamin A, D₂, D₃, E (dl-a-tocopherol, da-tocopherol, d-d-tocopherol) and their derivatives such as palmitic acid ascorbate, stearic acid ascorbate, dipalmitic acid ascorbate, acetic acid-dl-a-tocopherol, nicotinic acid dl-a-tocopherol vitamin E, dl-pantothenyl alcohol, D-pantothenyl alcohol, pantothenyl ethylether etc. including the lipid soluble vitamin used in examples of present invention are preferable and those can be obtained by conventional method such as microbial conversion method, purification method from the microbial cultivates, enzymatic method or chemical synthetic method.

Preferable peptide polymers are any one which can be mixed with cosmetic, however, collagen, hydrolysable collagen, gelatin, elastin, hydrolysable gelatin, keratin etc. including the peptide polymer used in examples of present invention are preferable.

Preferable polysaccharide polymers are any one which can be mixed with cosmetic, however, hydroxy ethyl cellulose, xanthin gum, hyaluronic acid Na, chondroitin sulfate or their salt (Na salt etc) and the like are preferable. For example, chondroitin sulfate or the salt thereof etc can be used by being purified from mammal or fishes ordinarily.

Preferable sphingolipid are any one which can be mixed with cosmetic, however, ceramide, pit-sphingosin, sphingo-lipopolysaccharide and the like are preferable. Sphingo-lipid can be obtained by being purified from mammal, fish, shellfish, yeast or plant etc in conventional method.

Preferable seaweed extract is any one which can be mixed with cosmetic, however, the extract of brown algae, red algae, green algae and the like or the purified carrageenan, alginic acid, arginic acid Na, K isolated therefrom are preferable. Algae extract can be obtained by being purified from seaweed in conventional method.

The cosmetic composition of the present invention may combine with other ingredients used in conventional cosmetic composition, if necessary, together with above described essential ingredient.

Preferable above described other ingredients may comprise oil ingredient, humectants, emollients, surfactants, organic or inorganic dye, organic powder, ultraviolet ray absorbing agent, preservatives, antiseptics, antioxidants, plant extract, pH controller, alcohol, pigments, perfumes, refrigerants, blood circulator, antihidrotic, distilled water etc.

Preferable oil ingredients may comprise ester oil, hydrocarbon oil, silicone oil, fluoride oil, animal oil, plant oil and so on.

Preferable ester oil described above may comprise glyceryl tri-2-ethyl hexanoic acid, cetyl 2-ethyl hexanoic acid, isopropyl myristic acid, butyl myristic acid, isopropyl palmitic acid, ethyl stearic acid, octyl palmitic acid, isocetyl isostearic acid, butyl stearic acid, ethyl linoleic acid, isopropyl linoleic acid, ethyl oleic acid, isocetyl myristic acid, isostearyl myristic acid, isostearyl palmitic acid, octyldodecyl myristic acid, isocetyl isostearic acid, diethyl sebasic acid, isopropyl adipic acid, isoalkyl neopetanoic acid, glyceryl tri(capryl, capric acid), trimethylopropane tri-2-ethyl hexanoic acid, trimethylopropane triisostearic acid, pentaerythritol tetra-2 ethyl hexanoic acid, cetyl caprylic acid, decyl lauric acid, hexyl lauric acid, decyl myristic acid, myristyl myristic acid, cetyl myristic acid, stearyl stearic acid, decyl oleic acid, cetyl licinoleic acid, isostearyl lauric acid, isotridecyl myristic acid, isocetyl palmitic acid, octyl stearic acid, isocetyl stearic acid, isodecyl oleic acid, octyldodecyl oleic acid, octyldodecyl linoleic acid, isopropyl isostearic acid, cetostearyl 2-ethyl hexanoic acid, stearyl 2-ethyl hexanoic acid, hexyl isostearic acid, ethylene glycol dioctanoic acid, ethylene glycol dioleic acid, propylene glycol dicapric acid, propylene glycol di(capryl, capric acid), propylene glycol dicaprylic acid, neopentylglycol dicapric acid, neopentylglycol dioctanoic acid, glyceryl tricaprylic acid, glyceryl triundecylic acid, glyceryl triisopalmitic acid, glyceryl triisostearic acid, octyldodecyl neopentanoic acid, isostearyl octanoic acid, octyl isononanoic acid, hexyldecyl neodecanoic acid, octyldodecyl neodecanoic acid, isocetyl isostearic acid, isostearyl isostearic acid, octyldecyl isostearic acid, polyglycerin oleanoic acid ester, polyglycerin isostearic acid ester, triisocetyl citric acid, triisoalkyl citric acid, triisooctyl citric acid, lauryl lactic acid, myristyl lactic acid, cetyl lactic acid, octyldecyl lactic acid, triethyl citric acid, acetyltriethyl citric acid, acetyl tributyl citric acid, trioctyl citric acid, diisostearyl maleic acid, di 2-ethylhexyl hydroxy stearic acid, 2-ethyl hexyl succinic acid, diisobutyl adipic acid, diisopropyl sebasinic acid, dioctyl sebacinic acid, cholesteryl stearic acid, cholesteryl isostearic acid, cholesteryl hydroxy stearic acid, cholesteryl hydroxy stearic acid, cholesteryl oleic acid, dihydrocholesteryl oleic acid, pitsteryl isostearic acid, pitsteryl oleic acid, isocetyl 12-stealoyl hydroxy stearic acid, stearyl 12-stealoyl hydroxy stearic acid, isostearyl 12-stealoyl hydroxy stearic acid.

Preferable hydrocarbon oil described above may comprise squalene, liquid paraffin, α-olefin oligomer, isoparaffin, ceresin, paraffin, liquid isoparaffin, polybuden, microcrystalline wax, vaselin and the like.

Preferable silicone oil may comprise polymethylsilicone, methylphenylsilicone, methylcyclopolysiloxane, octamethylpolysiloxane, decamethylpolysiloxane, dodecamethylcyclosiloxane, dimethyl siloxane-methyl cetyloxysiloxan copolymer, dimethyl siloxane-methyl stealoxysiloxane copolymer, alkyl modified silicone oil, amino modified silicone oil and the like.

Preferable fluoride oil can comprise perfluoropolyether and the like.

Preferable animal or plant oil can comprise avocado oil, almond oil, olive oil, sesame oil, rice husk oil, safflower oil, soy-bean oil, corn oil, rape oil, amygdalin oil, palm kernel oil, palm oil, pimaja oil, sunflower oil, fruite seed oil, cotton seed oil, coconut palm oil cucui nut oil, wheat embryo bud oil, rice embryo bud oil, sia butter, eveningprimrose oil, marker daymia nut oil, medo home oil, egg yolk oil, lanolin, hempseed oil, mink oil, orange ruppy oil, hohoba oil, carnawa wax, liquid lanolin, solid pimaja wax and the like.

Preferable humectants can comprise water-soluble low molecular humectants, lipophilic low molecular humectants, water-soluble polymer and lipid soluble polymer.

Specifically, preferable water soluble low molecular humectants can comprise cerin, glutamine, sorbitol, mannitol, pyrrolidone-carboxylic acid Na, glycerin, propylene glycol, 1,3-butylene glycol, ethylene glycol, polyethylene glycol (polymerization index. >2), polypropylene glycol (polymerization index>2), lactic acid, lactate salt and the like.

Preferable lipid soluble low molecular humectants can comprise cholesterol, cholesteryl ester and the like.

Preferable water soluble polymer can comprise carboxy vinyl polymer, poly asparaginic acid salt, tragacanth, xanthin gum, HMC (hydroxy methyl celluose), HEC (hydroxy ethyl celluose), HPC (hydroxy propyl celluose), carboxymethylcellulose, water soluble chitin, chitosan, dextrin and the like.

Preferable lipid soluble polymer can comprise polyvinylpyrrolidone-eicocene copolymer, polyvinylpyrrolidone-hexadecene copolymer, nitrocellulose, dextrin fatty acid ester, silicone polymer and the like.

Preferable emollients can comprise long chain acyl glutamic acid cholesteryl ester, cholesteryl hydroxy stearic acid, 12-hydroxy stearic acid, rogic acid, lanolin fatty acid cholesteryl ester and the like.

Preferable surfactant can comprise nonionic surfactants, anionic surfactants, cationic surfactants, ambivalent surfactants and the like.

Specifically, preferable non-ionic surfactants can comprise self-emulsified monostearic acid glycerin, propylene glycol fatty acid ester, glycerin fatty acid ester, polyglycerin fatty acid ester, sorbitan fatty acid ester, polyoxyethylene (POE) sorbitan fatty acid ester, POE sorbitan fatty acid ester, POE glycerin fatty acid ester, POE alkyl ether, POE fatty acid ester, POE solid pimaja oil, POE pimaja oil, POE-POP copolymer, POE-POP alkyl ether, polyether modified silicone, lauric acid alkanol amide, alkyl amine oxide, hydrogen addition soybean phospholipid and the like.

Preferable anionic surfactants can comprise fatty acid soap, a-acyl sulfonic acid salt, alkyl sulfonic acid salt, alkyl ally sulfonic acid, alkyl naphthalene sulfonic acid salt, alkyl sulfonic acid salt, POE alkylether sulfate salt, alkyl amide sulfate salt, alkyl phosphate salt, POE alkyl phosphate salt, alkylamide phosphate salt, alkyloylalkyl taurine salt, N-acyl-amino acid salt, POE alkyl ether carboxylic acid salt, alkyl sulfo succinic aid salt, alkyl sulfo-acetic acid salt, acylated hydrolysable collagen peptide salt, perfluoro alkyl phosphate ester and the like.

Preferable cationic surfactant can comprise alkyl trimethyl ammonium chloride, stearyl trimethyl ammonium chloride, stearyl trimethyl ammonium bromide, setostearyltrimethyl ammonium chloride, distearyl dimethyl ammonium chloride, stearyl dimethyl benzyl ammonium chloride, vehenyltrimethyl ammonium bromide, benzalkonium chloride, diethylamino ethyl amide stearic acid, dimethylaminopropyl amide stearic acid, lanolin derivatives quaternary ammonium and the like.

Preferable ambivalent surfactants can comprise carboxy betaine type, amide betaine type, hydroxy sulfo betaine type, phosphobetaine type, aminocarboxylic acid, imidazoline derivatives type, amide amine type and the like.

Preferable organic and inorganic dyes can comprise silicic acid, anhydrous silicic acid, magnesium silicic acid, talc, ceracyte, mica, caolin, bengala, clay, bentonite, titan film mica, oxy chlorine bismuth, zirconium oxide, magnesium oxide, zinc oxide, titan oxide, aluminium oxide, calcium sulfate, barium sulfate, magnesium sulfate, calcium carbonate, magnesium carbonate, ferrous oxide, chromium oxide, chromium hydroxide, calamine, carbon black and the complex thereof as an inorganic dyes; polyamide, polyester, polypropylene, polystyrene, polyurethane, vinyl resin, urea resin, phenol resin, fluoride resin, silicone resin, acryl resin, melamine resin, epoxy resin, polycarbonated resin, divinyl benzene-styrene copolymer, silk powder, cellulose, CI pigment yellow, CI pigment orange as an organic dyes; and their complex etc.

Preferable organic powder can comprise metal soap such as calcium stearate; alkyl phosphonate metal salt such as sodium zinc cetylic acid, zinc laurylic acid, calcium laurylic acid; acylamino acid polyvalent metal salt such as calcium N-lauroyl-b-alanine, zinc N-lauroyl-b-alanine, calcium N-lauroyl-glycine etc.; amide sulfonic acid polyvalent metal salt such as calcium N-lauroyl-taurine, calcium N-palmitoyl-taurine; N-acyl basic amino acid such as NE-lauroyl-L-lysine, Nε-palmitoyl-lysine, Na-palmitoyl ornitine, Na-lauroly arginine, hardened lanolin fatty acid acyl arginine and the like; N-acylpolypeptide such as N-lauroylglycyl glycine; a-amino fatty acid such as a-amino caprylic acid, a-amino lauric acid and the like; polyethylene, polypropylene, nylon, polymethylmetacrylate, polystyrene, divinylbenzene-styrene copolymer, ethylene tetrafluoride and so on.

Preferable ultraviolet absorbing agents can comprise paraaminobenzoic acid, paraamonoethyl benzoate, paraamino amyl benzoate, paraamino octyl benzoate, ethyleneglycol salicylate, phenyl salicylate, octyl salicylate, benzyl salicylate, butylphenyl salicylate, homomentyl salicylate, benzyl cinnamic acid, paramethoxy 2-ethoxy ethyl cinnamic acid, paramethoxy octyl cinnamic acid, diparamethoxy mono-2-ethylhexane glyceryl cinnamic acid, paramethoxy isopropyl cinnamic acid, diisopropyl-diisopropyl cinnamate ester mixture, urokanic acid, ethyl urokanic acid, hydroxy methoxy benzophenone, hydroxymethoxy benzophenone sulfonic acid and their salt, dihydroxy methoxy benzophenone, dihydroxy methoxy benzophenone disulfonate Na, dihydroxy benzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, 2-(2-hydroxy-5-methylphenyl) benzotriazole and the like.

Preferable preservatives can comprise hinokitiol, trichloric acid, trichlorohydroxydiphenylether, chlorohexidine glucuronate, phenoxyethanol, resorcine, isopropylmethylphenol, azulene, salicylic acid, zinc pilithione, bezalconium HCl, photosensitizer 301, mononitroguaiacol Na, undecylenic acid etc.

Preferable antioxidants can comprise butylhydroxyanisole, propyl gallate, ellisorbate and the like.

Preferable pH controller can comprise citric acid, sodium citrate, malic acid, sodium malate, fumaric acid, sodium fumaric acid, succinic acid, sodium succinic acid, sodium hydroxide, sodium hydrogen phosphate and the like.

Preferable alcohol can comprise cetyl alcohol etc.

Furthermore, other ingredient addable to above described component and the amount thereof is not limited within the scope of the purpose and effect of the present invention, however, it is preferable that the amount of the other ingredients ranges from 0.01 to 5%, more preferably, 0.01 to 3% in that of total composition.

The cosmetic composition of the present invention can be modified as a solution, emulsion, cohesive mixture etc.

Above described ingredients such as water-soluble vitamin, lipid soluble vitamin, peptide polymer, polysaccharide polymer, sphingolipid, sea weed extract and addable ingredients which can be added other than above described ingredients if necessary, can be obtained by conventional methods disclosed in the literature (Matsumoto Mithio; Manual for the development of transdermal applied preparation. Seisi Press, 1st Ed., 1985).

Inventive compounds of the present invention have no toxicity and adverse effect therefore, they can be used with safe.

It will be apparent to those skilled in the art that various modifications and variations can be made in the compositions, use and preparations of the present invention without departing from the spirit or scope of the invention.

The present invention is more specifically explained by the following examples.

However, it should be understood that the present invention is not limited to these examples in any manner.

### Advantageous Effects of Invention

As described above, the present inventors demonstrated that the anti-inflammatory effects of inventive combined composition is potent by accomplishing in vitro experiments such as the inhibitory test on the expression of cytokines involved in skin inflammation (RPLPO, TSLP, GM-CSF and IL-1beta) (Experimental Example 1); as well as in vivo experiments such as the inhibitory effect on the atopic dermatitis using by BALB/C mice (Experimental Example 2); the inhibition test of expression of various cytokines involved in skin inflammation (GADPH, TSLP, GM-CSF, IL-4, IL10, IL-13, IL-31 and IL-33) using test animal (Experimental Example 3); the inhibition test of TSLP cytokine expression using test animal (Experimental Example 4), therefore, it is confirmed that inventive combined extract is very useful in the alleviation or treatment of skin inflammation as a form of topical medicament or cosmetic composition.

### BRIEF DESCRIPTION OF DRAWINGS

FIG. 1 shows the dermatitis induced dorsal skin lesions treated with test sample (WIN), dexamethasone (DEX) and distilled water (DIW);

FIG. 2 shows the stained test results with H&E and toluidine blue (TB) on the dermatitis induced dorsal skin lesions treated with test sample (WIN), dexamethasone (DEX) and distilled water (DIW)

### BEST MODE FOR CARRYING OUT THE INVENTION

It will be apparent to those skilled in the art that various modifications and variations can be made in the compositions, use and preparations of the present invention without departing from the spirit or scope of the invention.

The present invention is more specifically explained by the following examples.

However, it should be understood that the present invention is not limited to these examples in any manner.

### Examples

The following Examples and Experimental Examples are intended to further illustrate the present invention without limiting its scope.

### Example 1. The Preparation of Inventive Combined Extract (1)

20 g of dried Longanae Arillus (Buyoung Yakup Co. Ltd.), 20 g of dried Ligustici Tenuissimi Rhizoma (Buyoung Yakup Co. Ltd.) and 20 g of dried Polygalae radix(Buyoung Yakup Co. Ltd.) were cut into small pieces, mixed with 6 fold volume (v/w) of 20% ethanol in water and the mixture was subjected to reflux extraction at 90±5° C. for 3 days. After filtration of the extract through filter paper (pore size, less than 10 μm) to remove the debris, the remaining debris was further extracted two times with 4 fold volume (v/w) of 20% ethanol in water and the extract was filtered with filter paper (pore size, less than 10 μm).

The collected extract was mixed with together and concentrated under vaccuo (16 21 brix) to afford concentrated extract. The concentrated extract was dried with freeze drying process and pulverized (less than 50 mesh) to obtain 20.5 g (powder as dried basis, yield 33.4%) of inventive combined extract (1) (designated as “WIN-1001X” hereinafter)

### Example 2-6. The Preparation of Inventive Combined Extract (2)-(6)

Excepting adopting different combined ratio as well as different solvents disclosed in Example 1, all the procedure was identical with those in Example 1 to obtain various inventive combined extract of Longanae Arillus (LA), Ligustici Tenuissimi Rhizoma (LT) and Polygalae radix (PR) i.e., inventive combined extract (2) to inventive combined extract (6) of the present invention, which are used as a test samples in following experiment.

TABLE 1
various kinds of combined extract

| | Sample weight (g) | | | | | Extract | | Final |
|---|---|---|---|---|---|---|---|---|
| Example | LA* | PR* | LT* | solvent* | name | weight | | yield |
| Example 2 | 10 | 5 | 50 | 10% EtOH | WIN-1002X | 16.6 | g | 25.6% |
| Example 3 | 20 | 50 | 5 | Water | WIN-1003X | 24.7 | g | 32.9% |
| Example 4 | 10 | 80 | 20 | 70% BuOH | WIN-1004X | 32.3 | g | 29.4% |
| Example 5 | 5 | 50 | 20 | 50% EtOH | WIN-1005X | 21.5 | g | 28.7% |
| Example 6 | 30 | 10 | 2 | hexane | WIN-1006X | 12.6 | g | 30.1% |

*Longanae Arillus (LA),
Ligustici Tenuissimi Rhizoma (LT),
Polygalae radix (PR)

### Experimental Example 1. Inhibitory Effect on Cytokine Expression (In Vitro)

In order to determine the anti-inflammatory activity of inventive extract, following inhibition test of cytokine expression using HaCaT cell was performed according to the procedure disclosed in the literature (Jeong et al., 2019, J. Invest. Dermatol., May; 139 (5): pp 1098-1109).

HaCaT cell (human epithelial keratinocyte cell, 300493, CLS) was inoculated into DMEM medium containing 10% Fetal bovine serum, 100 units/m1 of penicillin, 100n/m1 of streptomycin (D6429, Sigma-Aldrich Co. Ltd) and was incubated in the incubator (HERA cell 150i, Thermo Fisher Scientific Co. Ltd.) maintaining optimum humidity (85-95%) and 5% CO₂ atmosphere.

For performing gene expression test, the incubated cells were transferred to 12 wells and 50 ng/ml of TNF alpha (RC214-12, Biobasic Co. Ltd) was treated therewith for 1 hour to induce inflammatory response. Dexamethasone (200 nM, positive control, “DEX”, D4902, Sigma-Aldrich Co. Ltd.) and distilled water (negative control, “DIW”) were used as comparative controls.

1 hour after inducing the inflammation, 1 μg/ml of inventive extract prepared in Examples was treated with identical medium and subjected to incubation for 1 hour. After the incubation, RNA (FATRR-001, Favorgen) was extracted from the cell and cDNA was synthesized from the RNA using by cDNA synthesis kit (RRO36A, TAKARA). The polymerization reaction was performed using by the synthesized cDNA and Sybrgreen kit (RT500M, Enzynomics) and then Real-time-PCR was performed using by primers for various cytokines involved in skin inflammation (RPLPO, TSLP, GM-CSF and IL-1beta) as disclosed in Table 2.

TABLE 2
The used primers in RT-PCR method

| | | | Sequence |
|---|---|---|---|
| human* | direction | sequence | I. D |
| RPLP0 | forward | 5′- AGC CCA GAA CAC TGG TCT C-3′ | 1 |
| | reverse | 5′- ACT CAG GAT TTC AAT GGT GCC-3′ | 2 |
| TSLP | forward | 5′-TAT GAG TGG GAC CAA AAG TAC CG-3′ | 3 |
| | reverse | 5′-GGG ATT GAA GGT TAG GCT CTG G-3′ | 4 |
| GM-CSF | forward | 5′-TCC TGA ACC TGA GTA GAG ACA C-3′ | 5 |
| | reverse | 5′-TGC TGC TTG TAG TGG CTG G-3′ | 6 |
| IL-1β | forward | 5′-CTC CAG GGA CAG GAT ATG GA-3′ | 7 |
| | reverse | 5′-TCT TTC AAC ACG CAG GAC AG-3′ | 8 |

*abbreviation- RPLP0 (Ribosomal Protein Lateral Stalk Subunit P0); TSLP (thymic stromal lymphopoietin); GM(Granulocyte-macrophage)-CSF (colony stimulating factor); IL (interleukin)

As can be seen in Table 3 showing quantitative result of the RT-PCR, the test sample group treated with the inventive extract, sharply inhibited the expressed level of various cytokine involved in skin inflammation comparing with negative control group treated with distilled water (DIW) and it has been confirmed that the inhibitory activity of the test sample on the expression of various cytokine involved in skin inflammation is equivalent to that of positive control group treated with dexamethasone (DEX).

Accordingly, it has been confirmed that the various kind of inventive combined extract prepared in Examples 1-6 have potent inhibitory effect on skin inflammation.

TABLE 3
Inhibition effect on cytokine expression
TSLP

| — | TNFα | TNFα | TNFα | TNFα | TNFα | TNFα |
|---|---|---|---|---|---|---|
| — | DIW | WIN-1001X | WIN-1002X | WIN-1003X | WIN-1005X | Dex |
| 1 | 132.4692 | 47.43735 | 60.85783 | 48.9323 | 55.34286 | 52.49334 |
| 0.462769 | 26.91228 | 9.645089 | 24.95619 | 19.85678 | 26.59252 | 11.2336 |

GM-CSF

| | | | | | | |
|---|---|---|---|---|---|---|
| — | TNFα | TNFα | TNFα | TNFα | TNFα | TNFα |
| — | DIW | WIN-1001X | WIN-1002X | WIN-1003X | WIN-1005X | Dex |
| 1 | 4.473627 | 1.982161 | 2.069408 | 2.384771 | 1.917569 | 1.935997 |
| 0.111 | 0.817826 | 0.889233 | 0.326074 | 0.871501 | 0.599711 | 0.581338 |

IL-1β

| | | | | | | |
|---|---|---|---|---|---|---|
| — | TNFα | TNFα | TNFα | TNFα | TNFα | TNFα |
| — | DIW | WIN-1001X | WIN-1002X | WIN-1003X | WIN-1005X | Dex |
| 1 | 4.152715 | 1.407169 | 1.437399 | 2.064964 | 1.662578 | 1.080503 |
| 0.483565 | 1.087056 | 0.394622 | 0.1926 | 0.620225 | 0.193175 | 0.413136 |

### Experimental Example 2. Inhibitory Effect on Atopic Dermatitis (In Vivo)

To confirm the inhibitory effect of inventive extract on the atopic dermatitis, the animal model test using by mice, was performed according to the method disclosed in the reference (Li et al., Drug. Des. Devel. Ther., 2016, Feb. 19; 10:781-191).

100 μL of 0.15% (w/v) DNFB (2,4-dinitrofluorobenzene, D1529, Sigma-Aldrich Co. Ltd.) was spread on abdominal cavity of 6 weeks-old BALB/C female mouse (DBL Co., Ltd. Incheon, Korea) and after removing the central dorsal hair, 100 μL of 0.15% (w/v) DNFB (2,4-dinitrofluorobenzene, D1529, Sigma-Aldrich Co. Ltd.) was spread at every third day starting from at 7th day to 16th day to induce skin inflammation. The test sample (10 mg/ml of inventive extract WIN-1001X, prepared in Examples) as well as Dexamethasone (200 μM, positive control, “DEX”, D4902, Sigma-Aldrich Co. Ltd.) and distilled water (negative control, “DIW”) used as comparative controls were spread every starting from at 7th day to 16^{th} day of initial treatment day of DNFB.

For the purpose of comparing with test group, the identical test excepting using acetone instead of DNFB and DIW instead of test sample with the above procedure was performed.

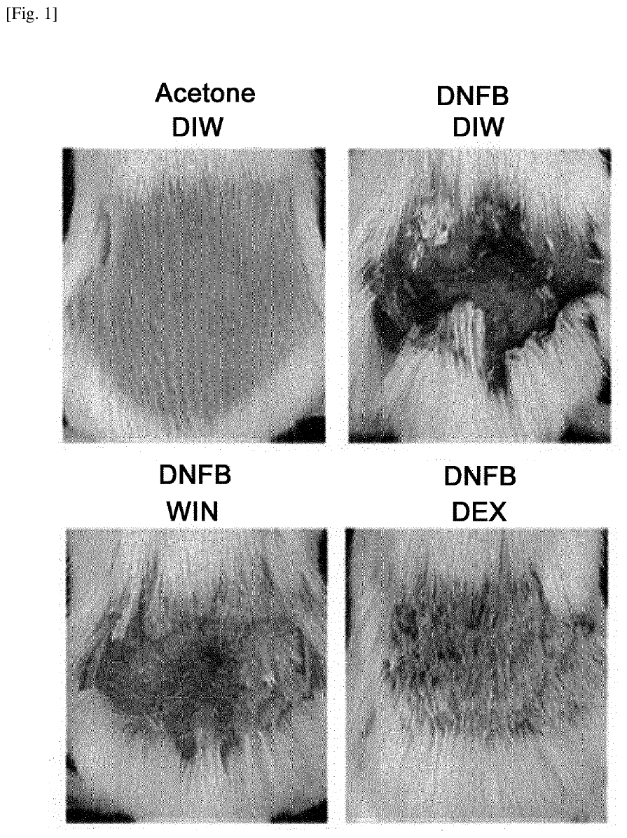

As can be seen in FIG. 1, it can be seen that skin dermatitis occurred on the dorsal skin of mice at 16th day after the sample treatment and the severity of skin dermatitis has been classified into four scores, i.e., score 0 (no syndrome), 1 (mere dermatitis), 2 (mean dermatitis) and 4 (severe dermatitis) with respect to four criteria on the syndrome, i.e., (i) erythema/bleeding, (ii) edema, (iii) abrasion/maceration, and (iv) scar/dryness to be total score 12.

As can be seen in FIG. 1 and Table 4, it has been confirmed that the inventive combined extract showed potent improving effect on skin dermatitis comparing with negative control group, of which effect is similar to that of positive control group treated with dexamethasone (DEX).

TABLE 4
Improving effect on skin dermatitis

| Acetone | DNFB | DNFB | DNFB |
|---|---|---|---|
| DIW | DIW | WIN-1001X | DEX |
| 0 | 8.8 | 5 | 2.2 |
| 0 | 1.30384 | 0.707107 | 0.83666 |

Additionally, the histological analysis on skin tissue of test animal has been performed.

Specifically, the dorsal skin tissue occurring dermatitis was fixed on 4% (w/v) paraformaldehyde solution (P6148, Sigma-Aldrich) in shaker maintaining 4° C. (CR300, FINEPCR) at 16th day after the sample treatment and 12 hours after the fixation, the dehydrated and transparent tissue was embedded in paraffin, sliced (5 μm of width) to make tissue slices.

The tissue slices were stained with H&E staining agent for staining skin epidermal layer {hematoxylin (S3309<DAKO) & Eosin (109844, Millipore)} or toluidine blue for staining mast cells infiltrated into inflamed lesion (TB, 185426, Sigma-Aldrich) and the result was observed using by microscope (EVOS XL., Life Technologies).

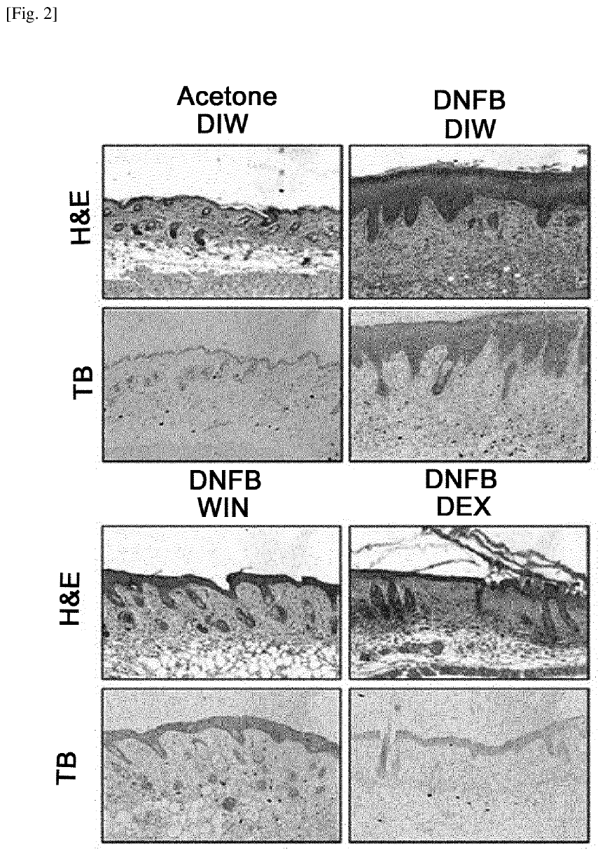

As can be seen FIG. 2 and Tables 5-6, it has been confirmed that the test group treated with inventive combined extract showed significantly reduced width of skin epidermal layer as well as reduced number of mast cell infiltrated into inflamed lesion comparing with negative control group treated with DIW, of which effect is similar to that of positive control group treated with dexamethasone (DEX).

TABLE 5
effect on the relative area of epidermis (fold)

| Acetone | DNFB | DNFB | DNFB |
|---|---|---|---|
| DIW | DIW | WIN-1001X | DEX |
| 1 | 7.06432606 | 3.55861484 | 1.638117 |
| 0.173221 | 2.07300369 | 0.42330352 | 0.875806 |

TABLE 6
effect on the relative number of mast cells (fold)

| Acetone | DNFB | DNFB | DNFB |
|---|---|---|---|
| DIW | DIW | WIN-1001X | DEX |
| 1 | 3.182796 | 1.72043 | 0.854839 |
| 0.312754 | 1.003359 | 0.263386 | 0.270532 |

### Experimental Example 3. Inhibitory Effect on Cytokine Expression (In Vivo)

In order to determine the anti-inflammatory activity of inventive extract, following inhibition test of cytokine expression using test animal was performed according to the procedure disclosed in the literature (Li et al., Drug. Des. Devel. Ther., 2016, Feb. 19; 10:781-191).

At 16th day after the treatment of DNFB, RNA (FATRR-001, Favorgan) was extracted from the dorsal skin of mice prepared in Experimental Example 2 and cDNA was synthesized from the RNA using by cDNA synthesis kit (RRO36A, TAKARA). The polymerization reaction was performed using by the synthesized cDNA and Sybrgreen kit (RT500M, Enzynomics) and then Real-time-PCR was performed using by primers for various cytokines involved in skin inflammation (GADPH, TSLP, GM-CSF, IL-4, IL-10, IL-13, IL-31 and IL-33) as disclosed in Table 7.

TABLE 7
The used primers in RT-PCR method

| | | | Sequence |
|---|---|---|---|
| mouse* | direction | sequence | I. D |
| GAPDH | forward | 5′- AGG TCG GTG TGA ACG GAT TTG-3′ | 9 |
| | reverse | 5′-TGT AGA CCA TGT AGT TGA GGT CA-3′ | 10 |
| TSLP | forward | 5′-AGC TTG TCT CCT GAA AAT CGA G-3′ | 11 |
| | reverse | 5′-AGG TTT GAT TCA GGC AGA TG TT-3′ | 12 |
| CSF2 | forward | 5′-AGG GTC TAC GGG GCA ATT TC-3′ | 13 |
| | reverse | 5′-TCA CAG TCC GTT TCC GGA GTT-3′ | 14 |
| IL-4 | forward | 5′-GGT CTC AAC CCC CAG CTA GT-3′ | 15 |
| | reverse | 5′-GCC GAT GAT CTC TCT CAA GTG AT-3′ | 16 |
| IL-10 | forward | 5′-GCT CTT ACT GAC TGG CAT GAG-3′ | 17 |
| | reverse | 5′-CGC AGC TCT AGG AGC ATG TG-3′ | 18 |
| IL-13 | forward | 5′-CCT GGC TCT TGC TTG CCT T-3′ | 19 |
| | reverse | 5′-GGT CTT GTG TGA TGT TGC TCA-3′ | 20 |
| IL-22 | forward | 5′-ATG AGT TTT TCC CTT ATG GGG AC-3′ | 21 |
| | reverse | 5′-GCT GGA AGT TGG ACA CCT CAA-3′ | 22 |
| IL-31 | forward | 5′-TCA GCA GAC GAA TCA ATA CAG C-3′ | 23 |
| | reverse | 5′-TCG CTC AAC ACT TTG ACT TTC T-3′ | 24 |
| IL-33 | forward | 5′-GCT GCA GAA GGG AGA AAT CAC G-3′ | 25 |
| | reverse | 5′-GGA GTT GGA ATA CTT CAT TCT AGG | 26 |
| | | TCT CAT-3′ | |

*abbreviation- GAPDH (Glyceraldehyde-3-phosphate dehydrogenase); TSLP (thymic stromal lymphopoietin); GM(Granulocyte-macrophage)-CSF (colony stimulating factor); IL (interleukin)

As can be seen in Table 8 showing quantitative result of the RT-PCR, the test sample group treated with the inventive extract, sharply inhibited the expressed level of various cytokine involved in skin inflammation comparing with negative control group treated with distilled water (DIW) and it has been confirmed that the inhibitory activity of the test sample on the expression of various cytokine involved in skin inflammation is equivalent to that of positive control group treated with dexamethasone (DEX).

Accordingly, it has been confirmed that the inventive combined extract prepared in Example has potent inhibitory effect on skin inflammation.

TABLE 8
Inhibition effect on cytokine expression
CSF2

| Acetone | DNFB* | DNFB | DNFB |
|---|---|---|---|
| DIW | DIW** | WIN-1001X | DEX*** |
| 1 | 72.14648 | 4.676082 | 7.817781 |
| 0.210396 | 12.96696 | 1.723294 | 0.98556 |

IL-4

| Acetone | DNFB | DNFB | DNFB |
|---|---|---|---|
| DIW | DIW | WIN-1001X | DEX |
| 1 | 11.36284 | 1.06316 | 4.798023 |
| 0.543902 | 3.453585 | 0.037212 | 2.312695 |

IL-10

| Acetone | DNFB | DNFB | DNFB |
|---|---|---|---|
| DIW | DIW | WIN-1001X | DEX |
| 1 | 19.29307 | 8.810186 | 20.44472 |
| 0.354329 | 1.973736 | 0.411741 | 4.157348 |

IL-13

| Acetone | DNFB | DNFB | DNFB |
|---|---|---|---|
| DIW | DIW | WIN-1001X | DEX |
| 1 | 48.62991 | 6.488924 | 12.56024 |
| 0.465986 | 10.37666 | 3.599525 | 8.866739 |

IL-22

| Acetone | DNFB | DNFB | DNFB |
|---|---|---|---|
| DIW | DIW | WIN-1001X | DEX |
| 1 | 29.18521 | 1.303359 | 13.08191 |
| 0.577444 | 2.801416 | 0.465745 | 1.945029 |

IL-31

| Acetone | DNFB | DNFB | DNFB |
|---|---|---|---|
| DIW | DIW | WIN-1001X | DEX |
| 1 | 3.933601 | 1.217765 | 1.000609 |
| 0.473134 | 0.44744 | 0.304217 | 0.13203 |

IL-33

| Acetone | DNFB | DNFB | DNFB |
|---|---|---|---|
| DIW | DIW | WIN-1001X | DEX |
| 1 | 9.471517 | 0.541047 | 0.736505 |
| 0.487178 | 2.797898 | 0.460806 | 0.486004 |

*DNFB: 2,4-dinitrofluorobenzene;
**DIW: distilled water;
***DEX: Dexamethasone

### Experimental Example 4. Inhibitory Effect on TSLP Cytokine Expression (In Vivo)

In order to determine the anti-inflammatory activity of inventive extract, following inhibition test of TSLP cytokine expression using test animal was performed according to the procedure disclosed in the literature (Li et al., Drug. Des. Devel. Ther., 2016, Feb. 19; 10:781-191).

At 16th day after the treatment of DNFB, RNA (FATRR-001, Favorgan) was extracted from the dorsal skin of mice prepared in Experimental Example 2 and cDNA was synthesized from the RNA using by cDNA synthesis kit (RRO36A, TAKARA). The polymerization reaction was performed using by the synthesized cDNA and Sybrgreen kit (RT500M, Enzynomics) and then Real-time-PCR was performed using by primers for TSLP cytokines involved in skin inflammation as disclosed in Table 7.

As can be seen in Table 9 showing quantitative result of the RT-PCR, the test sample group treated with the inventive extract, sharply inhibited the expressed level of TSLP cytokine involved in skin inflammation comparing with negative control group treated with distilled water (DIW) and it has been confirmed that the inhibitory activity of the test sample on the expression of TSLP cytokine involved in skin inflammation is equivalent to that of positive control group treated with dexamethasone (DEX).

Accordingly, it has been confirmed that the inventive combined extract prepared in Example has potent inhibitory effect on the expression of TSLP cytokine.

TABLE 9
Inhibition effect on TSLP cytokine expression
TSLP

| Acetone | DNFB | DNFB | DNFB |
|---|---|---|---|
| DIW | DIW | WIN-1001X | DEX |
| 1 | 7.514841 | 1.525431 | 1.310709 |
| 0.45284 | 1.954331 | 0.784039 | 0.32691 |

Statistics Analysis

The average and standard error were calculated from the test results obtained from the experiment. The significance difference test was analyzed using t-test, and the significance level (P-value) was expressed as P≤0.05=*, P≤0.01=**, and P≤0.001=***.

### MODE FOR THE INVENTION

Hereinafter, the formulating methods and kinds of excipients will be described, but the present invention is not limited to them. The representative preparation examples were described as follows.

Preparation of Skin Lotion

Extract of Example (WIN-1001X) 1.00%

Glycerol 3.00%

Ethanol 1.00%

Propylene glycol 0.10%

Flavour trace amount

Distilled water up to 100%

Skin preparation was prepared by dissolving the active components according to conventional lotion preparation method.

Preparation of Lotion

Extract of Example (WIN-1002X) 3.00%

L-ascorbic acid-2-magnesium phosphate 1.00%

Soluble collagen (1% solution) 1.00%

Sodium citric acid 0.10%

1,3-butylene glycol 3.00%

Distilled water up to 100%

Lotion preparation was prepared by dissolving the active components according to conventional lotion preparation method.

Preparation of Cream

Extract of Example (WIN-1003X) 3.00%

Polyethyleneglycomonosterate 2.00%

Monostearate glycerin 1.00%

Cetyl alcohol 4.00%

Squalene 6.00%

Tri 2-glycerly ethylhexanoate 6.00%

Sphingo-glycolipid 1.00%

1,3-butylene glycol 3.00%

Distilled water up to 100%

Cream preparation was prepared by dissolving the active components according to conventional cream preparation method.

Preparation of Pack

Extract of Example (WIN-1004X) 5.00%

Polyvinyl alcohol 13.00%

L-ascorbic acid-2-magnesium phosphate 1.00%

Lauroylhydroxyproline 1.00%

Soluble collagen (1% solution) 2.00%

1,3-butylene glycol 3.00%

Ethanol 5.00%

Distilled water up to 100%

Sugar 20 g

Fructose 20 g

Lemon flavor optimum amount

Distilled water 100 ml

Pack preparation was prepared by dissolving the active components according to conventional pack preparation method.

Preparation of Beauty Solution

Extract of Example (WIN-1005X) 2.00%

Hydroxyethylene cellulose (2% solution) 12.00%

Xanthin gum (2% solution) 2.00%

1,3-butylene glycol 3.00%

Glycerin concentration 4.00%

Sodium hyaluronte 5.00%

Distilled water 100 ml

Beauty solution preparation was prepared by dissolving the active components according to conventional beauty solution preparation method

The invention being thus described as will be obvious that it may be varied in many ways. Such variations are not to be regarded as a departure from the spirit and scope of the present invention, and all such modifications as would be obvious to those skilled in art are intended to be included within the scope of the following claims.

The invention being thus described, it will be obvious that the same may be varied in many ways. Such variations are not to be regarded as a departure from the spirit and scope of the present invention, and all such modifications as would be obvious to one skilled in the art are intended to be included within the scope of the following claims.

### INDUSTRIAL APPLICABILITY

As described in the present invention, the present invention provides a topical composition and cosmetic composition comprising a combined herb extract of Longanae Arillus, Ligustici Tenuissimi Rhizoma and Polygalae radix, the present inventors demonstrated that the anti-inflammatory effects of inventive combined composition is potent by accomplishing in vitro experiments such as the inhibitory test on the expression of cytokines involved in skin inflammation (RPLPO, TSLP, GM-CSF and IL-1beta) (Experimental Example 1); as well as in vivo experiments such as the inhibitory effect on the atopic dermatitis using by BALB/C mice (Experimental Example 2); the inhibition test of expression of various cytokines involved in skin inflammation (GADPH, TSLP, GM-CSF, IL-4, IL-10, IL-13, IL-31 and IL-33) using test animal (Experimental Example 3); the inhibition test of TSLP cytokine expression using test animal (Experimental Example 4), therefore, it is confirmed that inventive combined extract is very useful in the alleviation or treatment of skin inflammation as a form of topical medicament or cosmetic composition.

1. A topical pharmaceutical composition comprising a combined herb extract of Longanae Arillus Ligustici Tenuissimi Rhizoma and Polygalae radix as an active ingredient to inhibit TLSP (thymic stromal lymphopoietin) cytokine expression or to treat and alleviate skin inflammatory diseases.

2. The topical pharmaceutical composition according to claim 1, wherein said combined herb extract is (a) the combined herb extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w), or (b) the combination of each extract of Longanae Arillus, Ligustici Tenuissimi Rhizoma and Polygalae radix with the mixed ratio based on the dried weight of Longanae Arillus, Ligustici Tenuissimi Rhizoma and Polygalae radix (w/w) ranging from 0.01-100:0.01-100:0.01-100 weight part (w/w).

3. The topical pharmaceutical composition according to claim 1, wherein said extract is extracted with at least one solvent selected from water, C 1-C4 lower alkyl alcohol such as methanol, ethanol, propanol, butanol, etc, acetone, ethyl acetate, chloroform, hexane, butyleneglycol, propyleneglycol or glycerin.

4. The topical pharmaceutical composition according to claim 1, wherein said skin inflammatory diseases is the disease selected from group of pruritus caused by aging or atopy, chronic relapsing dermatitis such as atopic dermatitis, psoriasis, and the like; contact dermatitis, seborrheic dermatitis, neurodermatitis, xeroderma, erythema, inflammatory dermatitis, psoriasis, or atopic disease.

5. A method of inhibiting TLSP (thymic stromal lymphopoietin) cytokine, treating or alleviating skin inflammatory diseases in a mammal comprising topically administering to said mammal an effective amount of the combined herb extract of Longanae Arillus, Ligustici Tenuissimi Rhizoma and Polygalae radix and pharmaceutically acceptable carrier thereof.

6. (canceled)

7. (canceled)

8. A cosmetic composition comprising the combined herb extract of Longanae Arillus, Ligustici Tenuissimi Rhizoma and Polygalae radix as an active ingredient in an amount effective to inhibit TLSP (thymic stromal lymphopoietin) cytokine or to treat and alleviate skin inflammatory diseases.

9. The cosmetic composition according to claim 8, wherein said extract is extracted with at least one solvent selected from water, C1-C4 lower alkyl alcohol such as methanol, ethanol, propanol, butanol, etc, acetone, ethyl acetate, chloroform, hexane, butyleneglycol, propyleneglycol or glycerin.

10. The cosmetic composition according to claim 8, wherein said skin inflammatory diseases is the disease selected from group of pruritus caused by aging or atopy, chronic relapsing dermatitis such as atopic dermatitis, psoriasis, and the like; contact dermatitis, seborrheic dermatitis, neurodermatitis, xeroderma, erythema, inflammatory dermatitis, psoriasis, or atopic disease.

11. The cosmetic composition according to claim 8, wherein said composition is a form selected from skin lotion, skin softener, skin toner, astringent, lotion, milk lotion, moisture lotion, nutrient lotion, massage cream, nutrient cream, moisture cream, hand cream, foundation, essence, nutrient essence, pack, cleansing foam, cleansing lotion, cleansing cream, body lotion, body cleanser, treatment, beauty solution and the like.
